(19) United States
(12) Patent Application Publication
(10) Pub. No.: US 2007/0009473 A1
(43) Pub. Date: Jan. 11, 2007

(54) Vintage dated wine soap

(76) Inventor: Robert Wilford Walker, San Mateo, CA (US)

Correspondence Address: ROBERT WILFORD WALKER, 706 EDGEWOOD RD., SAN MATEO, CA, 94402, US

(21) Appl. No.: 11/177,747

(22) Filed: Jul. 8, 2005

(51) Int. Cl. A61K 8/97; A61K 36/87

(52) U.S. Cl. 424/74; 424/766

(57) ABSTRACT

This vintage dated wine soap is a hand made cleansing agent intended for use by humans and on certain animals. It combines vintage dated wine with various oils and fragrances in a cold blending process that preserves the characteristics of the wine, including the color, aromas and astringency. The glycerin which occurs naturally during production is left in the soap and not removed, as in most commercial soaps. It is an accepted fact in the wine industry that each vintage (harvest year) produces different flavors, colors and/or aromas in the grapes and therefore, in the wine from that vintage. Our ingredients and cold blending production process allows the character of the vintages and the wine to be reflected in the soap. I do not know of any other vintage dated wine soap.

### INVENTION

Vintage dated wine soap, for use by humans and/or animals, made with vintage dated wine and other ingredients.

### SPECIFICATION

1. This patent application is for soap produced for use on humans and/or animals, depending on the soap type.
2. The soap can be a solid or a liquid.
3. This vintage dated wine soap is produced by cold blending vintage dated wine, and various oils and fragrances in a 50-pound block, and then letting the soap cure and harden. The soap is then sliced into bars of various sizes. The bars are then wrapped in tissue paper or shrink-wrap, labeled, and shipped.
4. The soap recipes vary according to the soap type and include various oils, such as olive oil, palm oil and/or coconut oil, and other ingredients, in addition to vintage dated wine.

1. The invention claimed is for vintage dated soap made with vintage dated wine for use by humans and/or animals.

2. The invention claimed is a soap made with
A. A specified wine vintage (vintage means the year that the grapes used for that wine were harvested), and
B. A specified wine type (the type of grape or blended grapes, such as Chardonnay, Champagne, Sangria or Chablis) that gives the soap a distinctive character, color and fragrance.

3. The invention claimed is based on the fact that vintage dated wines have specific properties and characteristics, including but not limited to color and aroma, that give each vintage and wine type its specific characteristics.

4. The soap is produced using vintage dated wine as a key ingredient. The vintage of the wine gives the soap its vintage. In other words, if 2004 Chardonnay wine was used to make the soap, the soap vintage and type would be 2004 Chardonnay.

5. The wine used in the soap adds color, character and fragrance to the final product, and the color, character and fragrance varies depending on the wine used.

6. The vintage dated soap could also contain a specific wine brand. For example, 2004 Hess Chardonnay.

7. The patent application for vintage dated wine soap includes, but is not limited to, soaps with:
A. Varietal wine names, such as Chardonnay, Merlot, Cabernet, Cabernet Sauvignon, Cabernet Franc, Malbec, Pinot Noir, Pinotage, Sauvignon Blanc, Pinot Grigio, Pinot Gris, Zinfandel, White Zinfandel, etc.
B. Generic wine names, such as the American terms Chablis, Burgundy, sparkling wine, sherry, port, Sangria, vermouth, etc.
C. Appellation and place names, such as the French terms Chablis, Burgundy, Champagne, Beaujolais, etc.

8. This claim does not apply to wine soap that is non-vintage dated. For example, “Mary's Wine Soap”, or “Joe's Merlot Soap”.
